(19) United States
(12) Patent Application Publication
(10) Pub. No.: US 2022/0000389 A1
(43) Pub. Date: Jan. 6, 2022

(54) SIMULTANEOUS DETECTION OF PATHOGENS CAPTURED RESPECTIVELY FROM INHALATION AND EXHALATION

(71) Applicant: Amy Zhang Liu, Apex, NC (US)

(72) Inventor: Amy Zhang Liu, Apex, NC (US)

(21) Appl. No.: 16/921,749

(22) Filed: Jul. 6, 2020

(51) Int. Cl. A61B 5/08; C12Q 1/6888; G01N 21/76; A61B 5/097; A61B 5/00

(52) U.S. Cl. CPC A61B 5/082; C12Q 1/6888; G01N 21/76; A61B 5/097; A61B 2560/0431; A61B 5/7445; A61B 2560/0242; A61B 2560/0462; A61B 5/6819

(57) ABSTRACT

A methodology of detecting pathogens captured respectively from the inhalation and the exhalation in human breathing is disclosed. More particularly, a method is described for simultaneously detecting pathogens captured on sensors placed respectively inside an inhalation channel and an exhalation channel of a nose respirator.

### BACKGROUND OF THE INVENTION

### 1. Field of the Invention

The present invention relates to a methodology of detecting pathogens captured respectively from the inhalation and the exhalation in human breathing, more particularly to a method that can simultaneously detect pathogens captured on sensors placed respectively inside an inhalation channel and an exhalation channel of a nose respirator.

### 2. Description of Related Art

Human infection of pathogens via the respiratory system can result in various health problems. Respiratory infection of contagious pathogens presents a serious issue for public health and social safety. Detection of pathogens exposed and released by human beings is necessary for understanding epidemiology of the infection and finding ways to control the infection's spread.

Traditionally, pathogens exposed by human individuals are measured in various environmental samples. Pathogens released by human individuals are measured with samples taken from human bodies. However, information obtained from these two different types of sampling is often inadequate for determining if a specific individual is exposed to a pathogen because it is often unknown if this individual is present in the sampling environment at the sampling time. Examining human samples such as the swab of throat and the collection of washing fluid from respiratory system can show if a pathogen is present in the human body but cannot tell for sure if the pathogen will be released into environment by the human individual.

Recently it was claimed (https://www.medicaldesignandoutsourcing.com/this-face-masks-lights-up-when-it-detects-covid-19/) that pathogens in the exhaled air may be detected by a sensor embedded on the inside of a face mask. While the effectiveness of this approach is to be tested with planned experiments, some inherent limitations of this approach are obvious and may present drawbacks for its application.

The face mask-embedded sensor so far was intended for detecting only pathogens released by the person wearing a face mask. It is unknown if this approach would also be effective in detecting pathogens in the air to be inhaled by humans. Moreover, simultaneous detection of pathogens in inhaled air and in exhaled air seems difficult if not impossible with current sensor-embedded face mask.

### SUMMARY OF THE INVENTION

The primary objective of the present invention is to provide a methodology that can simultaneously detect pathogens captured from inhaled air and exhaled air respectively.

The secondary objective of the present invention is to provide a mechanism for allowing captured pathogens to be detected with sensors placed inside the inhalation channel and the exhalation channel respectively.

The third objective of the present invention is to provide a portable mechanism allowing for instant report of pathogen detection at any time/place.

Other objectives, advantages and novel features of the invention will become more apparent from the following description when taken in conjunction with the accompanying drawings.

### BRIEF DESCRIPTION OF THE DRAWINGS

FIG. 1A is a perspective view of a first embodiment of a set of pathogen-capturing devices to be used for the current invention along with a recently invented nose respirator device (U.S. patent application Ser. No. 16/868,538);

FIG. 1B is a bottom plan view of a pathogen-capturing device placed in the inner side of the filter in the exhalation channel of a nose respirator device shown in FIG. 1A;

FIG. 1C is a bottom plan view of a pathogen-capturing device placed on the outer side surface of the filter in the inhalation channel of a nose respirator device shown in FIG. 1A.

### DETAILED DESCRIPTION OF THE PREFERRED EMBODIMENT

The methodology in accordance with the present invention has three basic steps. The first step is to capture pathogens with a device placed in the inhalation channel and the exhalation channel respectively. The second step is to allow the pathogens to react with a sensor either embedded within the capturing device or placed inside a separator equipment. The third step is to detect the outcome of the reaction either in situ or a situ.

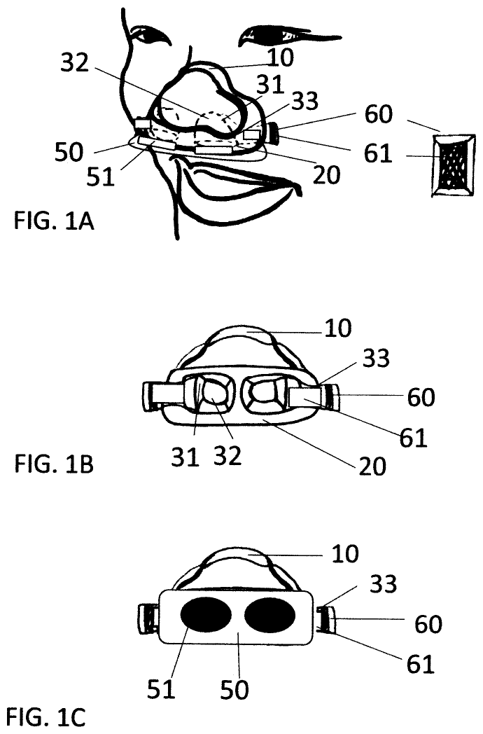

With reference to the series of FIGS. 1A to 1C, a first embodiment of a simultaneous detection of pathogens captured from respective inhaling and exhaling air in accordance with the present invention is shown, using capturing devices used together with a recently invented nose respirator device as an example. The nose respirator has a holder (10) connected to a base (20) which contains two cavities that each have an inhalation channel (32) and an exhalation channel (33) divided by a separator (31). A pathogen capturing device (60) is placed inside the exhalation channel on the inner sider of the filter so that the pathogen released by the exhaling air can be maximally captured and detected by a sensor (61) embedded within the capturing device. Another pathogen capturing device (50) is placed outside the filter in the inhalation channel so that the pathogen coming from inhaling air can be maximally captured and detected by a sensor (51) embedded within the capturing device.

Many different types of sensors may be used for detecting a pathogen captured. One example is paper-based RNA toehold sensors that were originally developed for detecting Zika virus (Pardee L. et al., 2016, Rapid, low-cost detection of Zika virus using programmable biomolecular components. Cell 165, 1255-1266) and recently adapted (https://www.medicaidesianandoutsourcing.com/this-face-masks-lights-up-when-it-detects-covid-19/) for detecting SARS-CoV-2, which is causing the ongoing pandemic of COVID-19. One advantage of this type of sensor is that it is stable at the normal temperature range of human life and thus can be used for the pathogen-capturing device disclosed in this patent.

The detection of captured pathogens can be done in different ways. Usually, it will require the captured pathogens to react with a sensor in equipment separate from the nose respirator. For example, the captured pathogens can be used as samples for PCR reactions aimed at detecting respiratory tract microbiota (RTM) (https://www.thermofisher.com/us/en/home/clinical/clinical-aenomics/pathogen-detection-solutions/real-time-pcr-respiratory-tract-microbiota-detection.html). However, the paper-based RNA toehold sensors may be improved so that their reaction with pathogens can be detected inside a nose respirator. For example, it is possible to detect the fluorescence inside the dark channel of a nose respirator if the pathogen-sensor reaction results in emission of fluorescence.

Simultaneous detection of pathogens from inhalation and exhalation respectively in individual human beings can provide valuable information regarding exposure, infection, and combination of them (Table 1).

TABLE 1
Sensor reaction results and indications

| Sensor reaction with | | |
|---|---|---|
| pathogens captured from | | |
| Inhalation | Exhalation | Indication |
| Positive | Negative | Normal person with exposure to pathogens |
| Negative | Positive | Infected person releasing pathogens |
| Positive | Positive | Infected person releasing pathogens and |
| | | exposed with pathogens again |
| Negative | Negative | No exposure to pathogens and no release |
| | | of pathogens |

Integrated with real-time recording of personal activities and linked with GIS-based tracking of human movement, a pathogen-capturing device attached to a nose respirator can yield rich information for comprehensive epidemiology studies. When big data of this information is effectively used, a human web of pathogen monitoring may be established for instant identification of hot environmental spots with high risk of pathogen exposure and timely reporting of infected individuals for immediate isolation and treatment.

Even though numerous characteristics and advantages of the present invention have been set forth in the foregoing description, together with details of the structure and function of the invention, that the disclosure is illustrative only, and changes may be made in details, especially in matters of shape, size, and arrangement of parts within the principles of the invention to the full extent indicated by the broad general meaning of the terms in which the appended claims are expressed is to be understood.

What is claimed is:

1. A methodology for detecting pathogens captured respectively in the inhalation air and the exhalation air of human breathing, having a step of:
a. Capturing pathogens from inhaling air and exhaling air respectively on a capturing device;
b. Allowing captured pathogens to react with a sensor; and
c. Reporting outcome of the reaction.

2. The method as claimed in claim 1, wherein the inhalation air and the exhalation air flow through an inhaling channel and an exhaling channel respectively in a nose respirator capable of separating these two air flows in human breathing.

3. The method as claimed in claim 1, wherein the sensor is a material that can react with a pathogen and release a detectable signal.

4. The method as claimed in claim 1, wherein the reporting of the outcome of the reaction is achieved through a mechanism of converting the reaction signal into reflection of the presence or absence of a pathogen.

5. The method as claimed in claim 3, wherein the sensor is embedded within a pathogen-capturing device.

6. The method as claimed in claim 4, wherein the reporting of the pathogen-sensor reaction is achieved with a portable device.

7. The method as claimed in claim 5, wherein the sensor embedded within a pathogen-capturing device reacts with the pathogen's specific nucleic acid and emits a signal.

8. The method as claimed in claim 7, wherein the sensor-emitted signal includes luminance and fluorescence.

9. The method as claimed in claim 6, wherein the reporting of the pathogen-sensor reaction is achieved with a portable fluorescence reader.

10. The method as claimed in claim 1, wherein the pathogen capture, the pathogen-sensor reaction and the reporting of the reaction result is done in situ on a nose respirator.
